(19) United States
(12) Patent Application Publication
(10) Pub. No.: US 2014/0066974 A1
(43) Pub. Date: Mar. 6, 2014

(54) MENSTRUAL PAIN TREATMENT DEVICE

(71) Applicant: Kyong Jun Park, Seoul (KR)

(72) Inventor: Kyong Jun Park, Seoul (KR)

(73) Assignees: COLOR SEVEN CO., LTD., Seoul (KR); Nam Gyun KIM, Jeonbuk (KR)

(21) Appl. No.: 14/118,226

(22) PCT Filed: Dec. 2, 2011
(86) PCT No.: PCT/KR2011/009289

§ 371 Date: Nov. 16, 2013

(30) Foreign Application Priority Data
May 16, 2011 (KR) 10-2011-0045559

(51) Int. Cl. A61N 5/06

(52) U.S. Cl. CPC A61N 5/0613
USPC 606/201; 607/90; 607/89

(57) ABSTRACT

The present invention relates to a menstrual pain treatment device, which is used bu being brought into close contact with a menstrual pain treatment area of the body or by being attached to a color light treatment area of the body using a double-sided adhesive tape or a hydro gel pad, and which comprises a pad- or patch-shaped main body for accommodating a battery and a PCB having a light source, a light source operation controller, and a treatment device operation button. The present invention treats menstrual pain by concentrating a colored light for treats menstrual pain by concentrating a colored light for treating menstrual pain on the menstrual pain treatment area of the body through the light emitting outlet in the patch-shaped main body. Since the present invention has the shape of a pad or patch, the present invention can be used by being attached to an underwear, a belt, or a pressurizing band, or by being brought into close contact with the menstrual pain treatment area of the body by being tied to that area eith a string. In addition, since the present invention, being in close contact with the menstrual pain treatment area of the body, concentrates a colored light having a wavelength range specified for the treatment of menstrual pain, the present invention can be expected to have a menstrual pain treatment effect, while completely eliminating the hygienic issues of a conventional menstrual pain treatment device inserted into the vagina.

### BACKGROUND

1. Field of the Invention

The present invention relates to a menstrual pain treatment device, and more particularly, to a menstrual pain treatment device in the form of a plate or a patch that is closely attached to a menstrual pain treatment site of a human body and intensively radiates color light with a predetermined wavelength range for the menstrual pain treatment.

2. Discussion of Related Art

A menstrual pain is a disease that occurs when women pay much attention to something or are under high stress. The pain occurring on a lower part of a belly and a waist when women start their menstruation is often referred to as the menstruation pain. However, the menstruation pain may also be accompanied by indigestion, emesis, headache, depression, and mental tension as well as backache and pantalgia.

As a treatment device for treating the menstrual pain described above, a menstrual pain treatment device is proposed in accordance with Korean Patent Publication No. 10-2001-0019454 (Publication Date: 15 Mar., 2001). This menstrual pain treatment device enables the blood to be normally circulated by being inserted into a womb to cause the womb to be unfolded, thereby effectively suppressing the menstrual pain.

However, the conventional menstrual pain treatment device mentioned above needs to be inserted into a vagina, which makes it inconvenient to use the menstrual pain treatment device and makes the vagina sanitarily weak.

### SUMMARY OF THE INVENTION

The present invention is intended to solve the conventional problem mentioned above, and it is an object of the present invention to provide a menstrual pain treatment device, which is used by being attached to underwear, a belt, a compression band, or the like, being tied with a string to be in close contact with a menstrual pain treatment site of a human body or being attached to a color light treatment site of the human body by virtue of with a double-sided adhesive tape or a hydrogel pad. The device includes a PCB having a light source (e.g., a Light Emitting Diode (LED), an Organic Light Emitting Diode (OLED), a Laser Diode (LD) or a three-color LED capable of emitting various color lights) emitting color light (e.g., orange, red, green, yellow or the like) with a wavelength range (e.g., a visible light wavelength band of 400 nm to 800 nm in which the color is visible when the color light reaches the skin) determined for treating the menstrual pain and a light source controller, a treatment device operating button connected to the PCB, and a main body in the form of a plate or a patch in which a battery is received. The device intensively radiates the color light for menstrual pain treatment onto a menstrual pain treatment site associated with a womb of the human body which is referred to in Oriental medicine (e.g., acupoints such as an acupoint Ki-Hae (Conception Vessel 6, CV6) acting to discharge carbonic acid gas by virtue of systematic circulation and activation and to relieve all pains below the navel or an acupoint Jung-Wan (Conception Vessel 12, CV12) acting to stimulate the brain activity to create an energy producing positive vitality and making metabolism smooth) for a predetermined time (e.g., 10±5 minutes) via a light-emitting hole penetrated in a front part of the main body to supply photon energy to damaged cells, thereby making cell metabolism smooth, promoting blood circulation, and enabling a sufficient amount of oxygen and nutrition to be supplied to treat the menstrual pain.

In order to achieve the object of the present invention described above, the menstrual pain treatment device of the present invention may include a main body in the form of a plate or a patch and divided into front and rear parts and of which edges are in contact with each other and sealed, a light-emitting hole being penetrated in the front part, and a transparent light diffusion film being attached to a rear surface of the light-emitting hole; a PCB received in a space between the front part and the rear part of the main body and including a light source (e.g., an LED, an OLED, or an LD), a treatment device operating button, and a light source operating controller, the light source emitting color light (e.g., orange, red, green, yellow or the like) with a predetermined wavelength range (e.g., a visible light wavelength band of 400 nm to 800 nm in which the color is visible when the color light reaches the skin) for menstrual pain treatment, the treatment device operating button outputting a treatment device operating signal whenever the button is pressed one time, the light source operating controller outputting a treatment start signal to turn on the light source for a predetermined time (e.g., 10±5 minutes) when the treatment device operating signal is input and automatically outputting a treatment end signal to turn off the light source when a predetermined time (e.g., 10±5 minutes) is elapsed, the light source emitting the color light via the light diffusion film and the light-emitting hole of the main body, and the treatment device operating button being disposed to be exposed outside the main body; and a battery received in the space between the front part and the rear part of the main body, being disposed between the PCB and the rear part, and supplying a driving power to the light source, the treatment device operating button, and the light source operating controller of the PCB.

The main body of the menstrual pain treatment device according to the present invention may be made of any one of cotton, rubber, PVC, silicon, urethane and may be in the form of a plate or a patch.

The light source of the menstrual pain treatment device according to the present invention may have at least one of any one of an LED, an OLED, an LD or a three-color LED capable of emitting various color lights.

The wavelength range of the color light emitted for the menstrual pain treatment by the light source of the menstrual pain treatment device according to the present invention may be a visible light wavelength band (e.g., 400 nm to 800 nm) in which the color is visible when the color light reaches a skin.

The light source operating controller of the menstrual pain treatment device according to the present invention may immediately output the treatment end signal to turn off the light source when the treatment device operating signal is input while the treatment start signal is output.

The battery of the menstrual pain treatment device according to the present invention may be any one of a thin-film type polymer battery and a coin type battery.

A double-sided adhesive tape or a hydrogel pad of which a hole is penetrated in its central portion and attached to a surface of the front part of the main body may be used so as to expose the light-emitting hole of the front part, so that the light-emitting hole of the front part of the main body may be in close contact with a menstrual pain treatment site of the human body.

According to the present invention, the menstrual pain treatment device is in the form of a plate or patch, which facilitates attaching the device to or tying the device with underwear, a belt, a compression band, or the like to cause the device to be in close contact with a menstrual pain treatment site of a human body. In particular, since the menstrual pain treatment device intensively radiates color light with a wavelength range determined for menstrual pain treatment while in close contact with the menstrual pain treatment site of the human body, the unsanitary problem of the conventional menstrual pain treatment device to be inserted into the vagina can be completely solved out and the menstrual pain treatment can be expected.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a disassembled perspective view illustrating a menstrual pain treatment device in accordance with the present invention;

FIG. 2 is a partial cross-sectional view of FIG. 1;

FIG. 3 is a view illustrating an embodiment in which the menstrual pain treatment device according to the present invention is attached to underwear and used;

FIG. 4 is a view illustrating an embodiment in which the menstrual pain treatment device according to the present invention is attached to a belt and used;

FIG. 5 is a view is a view illustrating an embodiment in which the menstrual pain treatment device according to the present invention is attached to a compression band and used;

FIG. 6 is a view illustrating an embodiment in which the menstrual pain treatment device according to the present invention is tied and used; and

FIG. 7 is a view illustrating an embodiment in which a double-sided adhesive tape or a hydrogel pad is attached to a front part of the menstrual pain treatment device according to the present invention.

### DETAILED DESCRIPTION OF EXEMPLARY EMBODIMENTS

Exemplary embodiments of the present invention will be described in detail below with reference to the accompanying drawings. While the present invention is shown and described in connection with exemplary embodiments thereof, it will be apparent to those skilled in the art that various modifications can be made without departing from the spirit and scope of the invention.

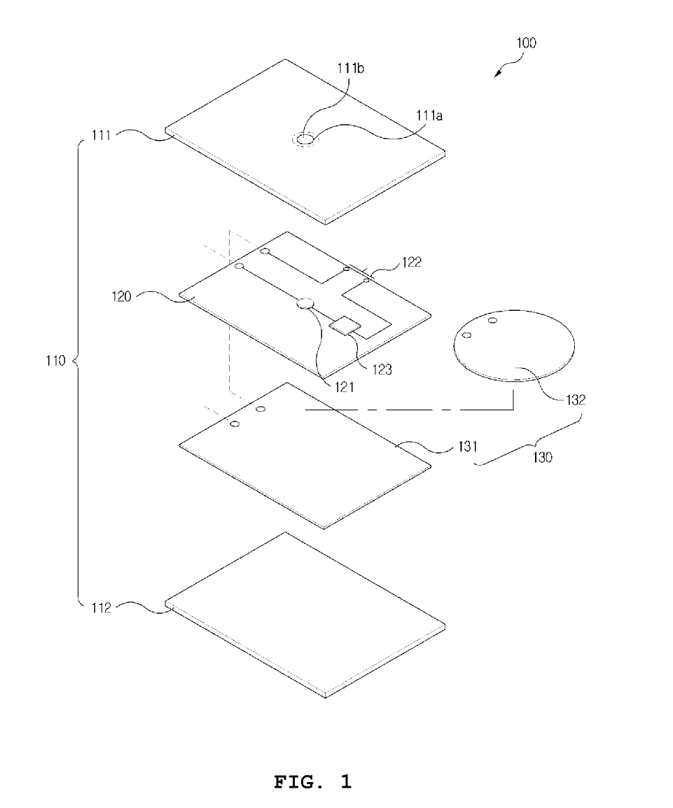

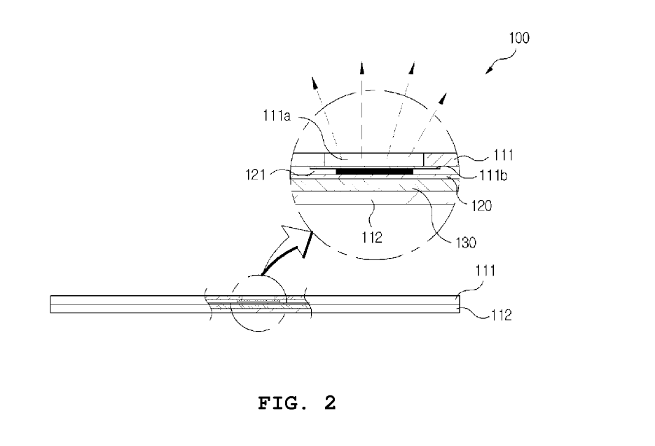

Referring to FIGS. 1 and 2, the menstrual pain treatment device 100 according to the present invention includes a main body 110, and a PCB 120 and a battery 130 which are received within the main body 110.

The main body 110 is in the form of a plate or a patch and is divided into front and rear parts 111 and 112 of which edges are in contact with each other and sealed. A light-emitting hole 111a is penetrated in the front part 111, and a transparent light diffusion film 111b is attached to a rear surface of the light diffusion hole 111a.

The main body 110 is made of any one of cotton, rubber, PVC, silicon, and urethane, and is in the form of the plate or the patch.

The shape of the main body 110 may be changed to various shapes such as a flat circle, a flat ellipse, a flat heart, or the like.

The PCB 120 is received in a space between the front part 111 and the rear part 112 of the main body 110, and includes a light source 121 (e.g., an LED, an OLED, an LD or a three-color LED capable of emitting various color lights) emitting color light (e.g., orange, red, green, yellow or the like) with a wavelength range (e.g., a visible light wavelength band of 400 nm to 800 nm in which the color is visible when the color light reaches the skin) determined for treating the menstrual pain, a treatment device operating button 122 outputting a treatment device operating signal whenever the button is pressed one time, and a light source operating controller 123 that outputs a treatment start signal to turn on the light source 121 for a predetermined time (e.g., 10±5 minutes) when the treatment device operating signal is input and automatically outputs a treatment end signal to turn off the light source 121 when a predetermined time (e.g., 10±5 minutes) is elapsed.

The light source 121 emits the color light via the light diffusion film 111b and the light-emitting hole 111a of the main body 110.

The light source 121 is preferably equipped with at least one of any one of an LED, an OLED, an LD, and a three-color LED.

The wavelength range of the color light emitted by the light source 121 for the menstrual pain treatment is preferably the visible light wavelength band (e.g., 400 nm to 800 nm) in which the color becomes visible when the color light reaches the skin.

The treatment device operating button 122 is exposed to a side of the main body 110.

The light source operating controller 123 immediately outputs the treatment end signal to turn off the light source 121 when the treatment device operating signal is input while the treatment start signal is output. The user can thus terminate the treatment by pressing the treatment device operating button 122 at any time if desired during the color light treatment.

The battery 130 is received in the space between the front part 111 and the rear part 112 of the main body 110, is disposed between the PCB 120 and the rear part 112, and supplies the driving power to the light source 121, the treatment device operating button 122, and the light source operating controller 123 of the PCB 120.

Any one of a thin-film type polymer battery 131 and a coin type battery 132 is preferably used as the battery 130.

The menstrual pain treatment device 100 of the present invention as configured above is used as follows.

The user may attach the menstrual pain treatment device 100 of the present invention to or tie the device with underwear, a belt, a compression belt, or the like to make the device in close contact with a menstrual pain treatment site of the body associated with the womb which is referred to in Oriental medicine (e.g., acupoints such as an acupoint QuiHai acting to discharge carbonic acid gas by virtue of systematic circulation and activation and to relieve all pains below the navel or an acupoint Zhong Wan acting to stimulate the brain activity to create an energy producing positive vitality and making metabolism smooth).

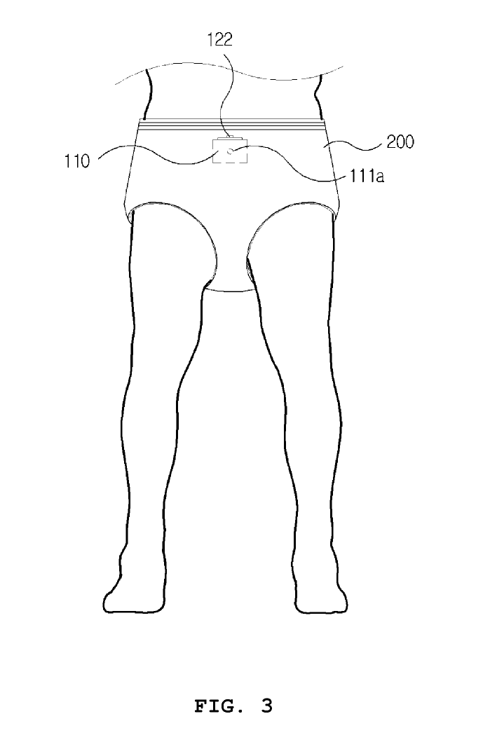

For example, the user may attach the menstrual pain treatment device 100 to the underwear so that the device is in close contact with the menstrual pain treatment site of the body as shown in FIG. 3. In this case, the user may preferably put on the underwear 200 such that the double-sided adhesive tape, the Velcro tape, or the adhesive is attached to a surface of the rear part 112 of the main body 110 or a rivet or a fixation pin is used to attach the menstrual pain treatment device 100 to the underwear 200 and the light-emitting hole 111a of the front part 111 of the main body 110 is thus in close contact with the menstrual pain treatment site of the human body.

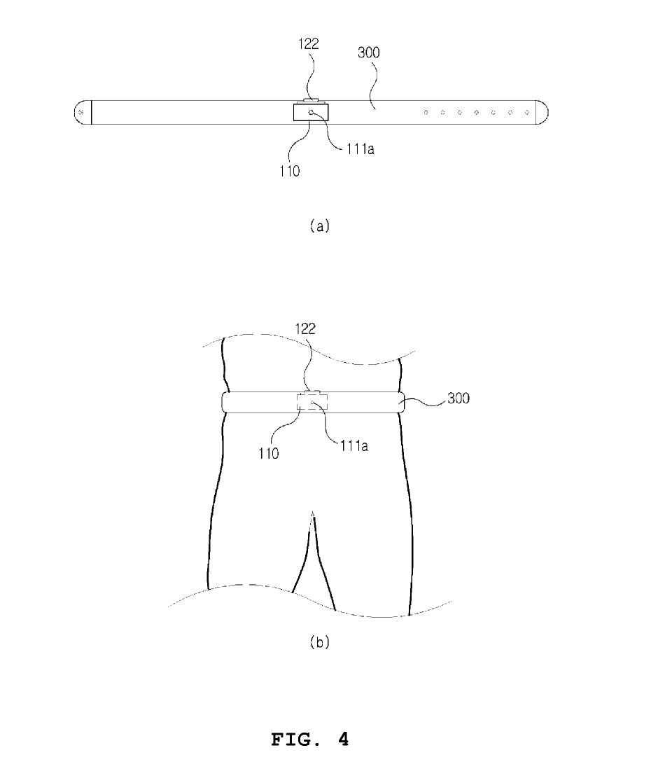

Alternatively, the user may closely attach the menstrual pain treatment device 100 to the menstrual pain treatment site of the human body as shown in FIG. 4B after attaching the device to the belt 300 as shown in FIG. 4A. In this case, the user may preferably put on the belt 300 such that the double-sided adhesive tape, the Velcro tape, or the adhesive is attached to a surface of the rear part 112 of the main body 110 or a rivet or a fixation pin is used to attach the menstrual pain treatment device 100 to the belt 300 and the light-emitting hole 111a of the front part 111 of the main body 110 is thus in close contact with the menstrual pain treatment site of the human body.

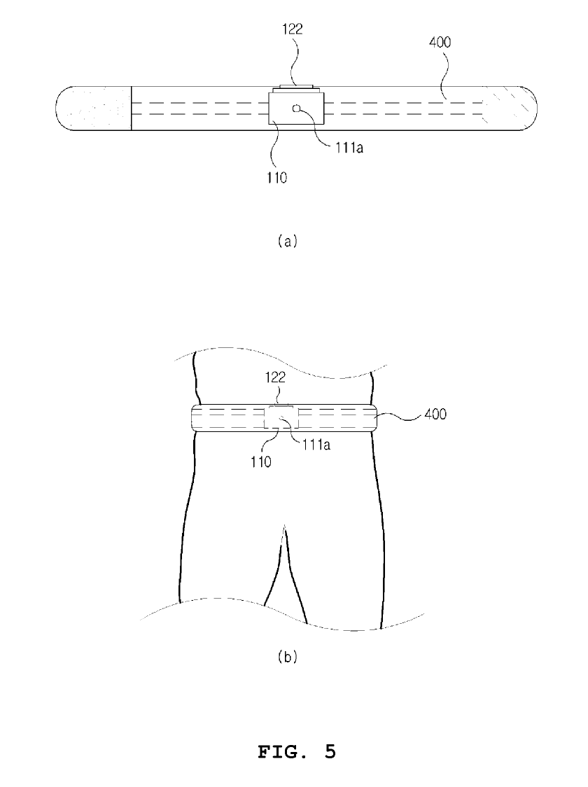

Alternatively, the user may closely attach the menstrual pain treatment device 100 to the menstrual pain treatment site of the human body as shown in FIG. 5B after attaching the device to the compression band 400 as shown in FIG. 5A. In this case, the user may preferably put on the compression band 400 such that the double-sided adhesive tape, the Velcro tape, or the adhesive is attached to a surface of the rear part 112 of the main body 110 or a rivet or a fixation pin is used to attach the menstrual pain treatment device 100 to the compression band 400 and the light-emitting hole 111a of the front part 111 of the main body 110 is thus in close contact with the menstrual pain treatment site of the human body.

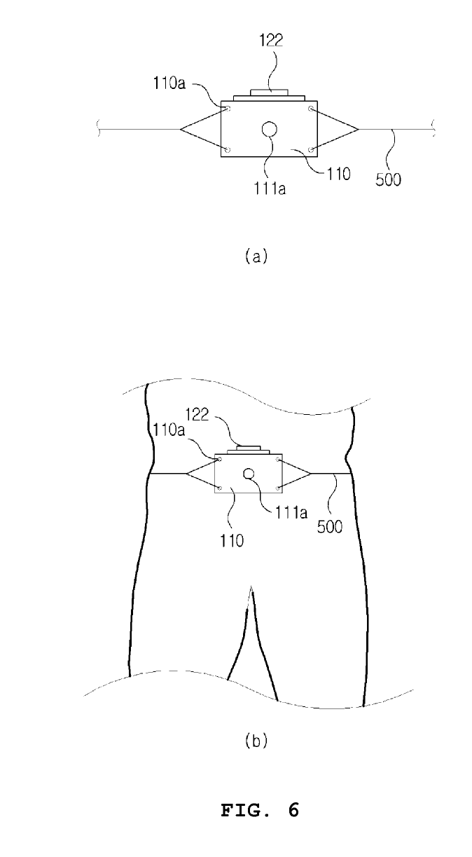

Alternatively, the user may closely attach the menstrual pain treatment device 100 to the menstrual pain treatment site of the human body as shown in FIG. 6B after tying the device 100 with the string 500 as shown in FIG. 6A. In this case, the user may preferably tie the human body with the string 500 such that the holes 110a penetrated in edges of the main body 110 are tied with the string 500 and the light-emitting hole 111a of the front part 111 of the main body 110 is thus in close contact with the menstrual pain treatment site of the human body.

When the user presses the treatment device operating button 120 while the user closely attaches the menstrual pain treatment device 100 of the present invention to the underwear 200, the belt 300, or the compression band 400, or ties the device with the string 500 to closely attach the device to the menstrual pain treatment site of the human body, the treatment device operating button 122 outputs the treatment device operating signal.

The light source operating controller 123 then outputs the treatment start signal to turn on the light source 121 for a predetermined time (e.g., 10±5 minutes) when the treatment device operating signal is input to the light source operating controller 123, and automatically outputs the treatment end signal to turn off the light source 121 when a predetermined time is elapsed.

When the light source 121 is turned on, the color light for treating the menstrual pain emitting via the light diffusion film 111b and the light-emitting hole 111a of the main body 110 is intensively radiated onto the menstrual pain treatment site for a predetermined time (e.g., 10±5 minutes) from that moment. As a result, a photon energy is supplied to damaged cells to make cell metabolism smooth, promote blood circulation, and enable a sufficient amount of oxygen and nutrition to be supplied to damaged womb tissues, thereby treating the menstrual pain.

The principle by which the menstrual pain is treated by the menstrual pain treatment device 100 of the present invention is as follows. Photons of a light energy intensively radiated onto the menstrual pain treatment site of the human body are moved along the meridian system to reach the womb, and then interact with photoreceptors in cells to increase creation of nitric oxide (NO) and concentration of cyclic Adenosine Monophosphate (cAMP) and relax the smooth muscle of the womb, thereby making the blood circulation smooth and the sufficient amount of oxygen and nutrition supplied to damaged womb tissues.

In the meantime, when the treatment device operating signal is output from the treatment device operating button 122 by the user pressing the treatment device operating button 122 and is input to the light source operating controller 123 while the light source 121 is turned on as described above, the light source operating controller 123 immediately outputs the treatment end signal to turn off the light source 121 while the treatment start signal is output. Accordingly, the user may terminate the treatment at any time if desired by pressing the treatment device operating button 122 while the menstrual pain is treated using the color light.

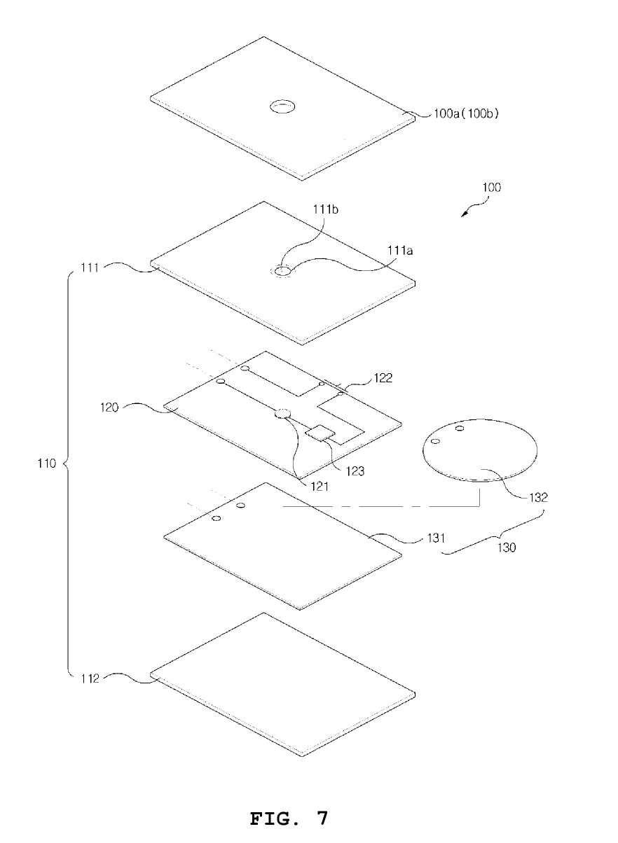

FIG. 7 is a view illustrating an embodiment in which the double-sided adhesive tape 100a, the hydrogel pad 100b or the like is attached to the front part 111 of the menstrual pain treatment device 100 according to the present invention.

In this case, the double-sided adhesive tape 100a, the hydrogel pad 100b or the like of which a hole is penetrated in its central portion is attached to a surface of the front part 111 of the main body 110 is used so as to expose the light-emitting hole 111a of the front part 111 is exposed. The light-emitting hole 111a of the front part 111 of the main body 110 is thus in close contact with the treatment site of the human body for the color light treatment.

The double-sided adhesive tape 100a, the hydrogel pad 100b or the like may be separately prepared by the user and then directly attached to the front part 111, or may be provided to the user in a state in which the double-sided adhesive tape, the hydrogel pad or the like is attached to the front part 111 in advance at the time of manufacturing the menstrual pain treatment device 100.

The menstrual pain treatment device of the present invention described above is not limited to the embodiments described above, and its technical spirit lies within the range in which the menstrual pain treatment device may be variously changed by those skilled in the art without departing from the scope of the claimed invention

What is claimed is:

1. A menstrual pain treatment device comprising:
a main body (110) in the form of a plate or a patch and divided into front and rear parts (111 and 112) of which edges are in contact with each other and sealed, a light-emitting hole (111a) being penetrated in the front part (111), and a transparent light diffusion film (111b) being attached to a rear surface of the light-emitting hole (111a);
a PCB (120) received in a space between the front part (111) and the rear part (112) of the main body (110) and including a light source (121), a treatment device operating button (122), and a light source operating controller (123), the light source emitting color light with a predetermined wavelength range for menstrual pain treatment, the treatment device operating button (122) outputting a treatment device operating signal whenever the button is pressed one time, the light source operating controller (123) outputting a treatment start signal to turn on the light source (121) for a predetermined time when the treatment device operating signal is input and automatically outputting a treatment end signal to turn off the light source (121) when a predetermined time is elapsed, the light source (121) emitting the color light via the light diffusion film (111b) and the light-emitting hole (111a) of the main body (110), and the treatment device operating button (122) being disposed to be exposed outside the main body (110); and
a battery (130) received in the space between the front part (111) and the rear part (112) of the main body (110), being disposed between the PCB (120) and the rear part (112), and supplying a driving power to the light source (121), the treatment device operating button (122), and the light source operating controller (123) of the PCB (120).

2. The menstrual pain treatment device of claim 1, wherein the main body (110) is made of any one of cotton, rubber, PVC, silicon, urethane and is in the form of a plate or a patch.

3. The menstrual pain treatment device of claim 1, wherein the light source (121) has at least one of any one of a Light Emitting Diode (LED), an Organic Light Emitting Diode (OLED), a Laser Diode (LD) or a three-color LED capable of emitting various color lights.

4. The menstrual pain treatment device of claim 1, wherein the wavelength range of the color light emitted for the menstrual pain treatment by the light source (121) is a visible light wavelength band in which the color is visible when the color light reaches a skin.

5. The menstrual pain treatment device of claim 1, wherein the light source operating controller (123) immediately outputs the treatment end signal to turn off the light source (121) when the treatment device operating signal is input while the treatment start signal is output.

6. The menstrual pain treatment device of claim 1, wherein the battery (130) is any one of a thin-film type polymer battery (131) and a coin type battery (132).

7. The menstrual pain treatment device of claim 1, wherein the main body (110) is used with a double-sided adhesive tape, a Velcro tape, or an adhesive attached to a surface of the rear part (112) or with a rivet or a fixation pin to make the rear part (112) of the main body (110) attached to any one of underwear (200), a belt (300) and a compression band (400), and the one of the underwear (200), the belt (300) and the compression band (400) is put on to make the light-emitting hole (111a) of the front part (111) of the main body (110) in close contact with a menstrual pain treatment site of a human body.

8. The menstrual pain treatment device of claim 1, wherein the light-emitting hole (111a) of the front part (111) of the main body (110) is in close contact with a menstrual pain treatment site of a human body when a string (500) is tied with holes (110a) penetrated in edges of the main body (110) and is then tied with a human body.

9. The menstrual pain treatment device of claim 1, wherein a double-sided adhesive tape (100a) or a hydrogel pad (100b) of which a hole is penetrated in its central portion and attached to a surface of the front part (111) of the main body (110) is used so as to expose the light-emitting hole (111a) of the front part (111), so that the light-emitting hole (111a) of the front part (111) of the main body (110) is in close contact with a menstrual pain treatment site of a human body.
